(19) United States
(12) Patent Application Publication
(10) Pub. No.: US 2010/0179358 A1
(43) Pub. Date: Jul. 15, 2010

(54) PROCCESS OF OXIDATIVE DEHYDROGENATION USING A BORIA-ALUMINA CATALYST

(76) Inventors: Yahia Al-Hamed, Jeddah (SA); Abdulrahim Al-Zahrani, Jeddah (SA); Mohammad Daous, Jeddah (SA); Khalid M. El-Yahyaoui, Meknes (MA)

Correspondence Address: SABIC AMERICAS, INC., 1600 INDUSTRIAL BLVD., SUGAR LAND, TX, 77478, US

(21) Appl. No.: 12/451,110

(22) PCT Filed: May 22, 2008
(86) PCT No.: PCT/EP2008/004098

§ 371 Date: Apr. 1, 2010

(30) Foreign Application Priority Data
May 23, 2007 (EP) 07010229.8

(51) Int. Cl. C07C 5/333; B01J 21/02

(52) U.S. Cl. 585/443; 585/440; 502/202

(57) ABSTRACT

The invention relates to a process of oxydehydrogenating an alkyl-substituted aromatic hydrocarbon starting compound into the corresponding alkenyl-substituted aromatic hydrocarbon product, respectively, which process comprises a step of contacting the starting compound and an oxidant at dehydrogenating conditions, in the presence of a boria-alumina catalyst, characterized in that the boria-alumina catalyst has been prepared by a co-precipitation method. The co-precipitation method comprises the steps of preparing a solution of aluminium salt in an organic medium, followed by adding to this solution a boron compound and then adding ammonia gas to the mixture obtained in previous step to form a precipitate and/or a gel. This process enables oxydehydrogenation of ethyl-benzene to styrene with high selectivity.

The invention relates to a process of oxidative dehydrogenation of an alkyl-substituted aromatic hydrocarbon starting compound into the corresponding alkenyl-substituted aromatic hydrocarbon product, which process comprises a step of contacting the starting compound and an oxidant at dehydrogenating conditions in the presence of a boria-alumina catalyst prepared by a co-precipitation method. More specifically, the invention relates to a process of oxidative dehydrogenation of ethyl-benzene to styrene. The invention further relates to a co-precipitation method of making said boria-alumina catalyst.

Such a process is known from publication W. Kania, M. Sopa—“Oxidative dehydrogenation of ethyl-benzene to styrene and modified alumina”, Polish J. Chem, 67 (1993), 419-423, which discloses a process to produce styrene by oxidative dehydrogenation (also called oxydehydrogenation) of ethyl-benzene, in the presence of a boron-modified aluminium oxide catalyst, hereinafter referred to as boria-alumina catalyst, having an atomic boron to aluminium ratio of 0.1 to 0.15. The catalyst was prepared via an impregnation method of preformed alumina with appropriate acids (e.g. boric acid) and then calcined at 500° C. for 4 hours.

Styrene is a very important aromatic hydrocarbon compound and is widely used as a raw material and a monomer for synthetic rubber, ABS resin and polystyrene. Conventionally, styrene is industrially manufactured by non-oxidative dehydrogenation of ethyl-benzene via excess steam over an iron oxide-based catalyst at about 600° C., giving a conversion of about 60% and a selectivity of about 90%. Alternatively, the oxidative dehydrogenation of ethyl-benzene (ODEB) to styrene is a known reaction in the prior art. Oxidative dehydrogenation, in which a hydrocarbon is reacted with molecular oxygen, enables in contrast to the non-oxidative dehydrogenation a virtually quantitative conversion to be achieved.

A large number of catalysts for the oxydehydrogenation of alkyl-substituted aromatic compounds to the corresponding alkenyl-substituted aromatics has been used in the prior art including phosphate, alumina, vanadium and carbon based catalysts, carbon supported catalysts or metal doped amorphous titanium oxide catalysts. For instance, U.S. Pat. No. 4,255,283 discloses the use of a metal phosphate, as catalyst. U.S. Pat. No. 3,497,564 teaches the use of carbon supported on an inorganic solid as oxydehydrogenating catalyst. Further, U.S. Pat. No. 5,895,829 directs to the use of a reducible metal oxide selected from the group consisting of V, Cr, Mn, Fe, Co, Pb, Bi, Mo, U and Sn, applied to carriers comprising clays, zeolites and oxides of Ti, Zr, Zn, Th, Mg, Ca, Ba, Si and Al. U.S. Pat. No. 4,652,690 discloses molecular sieve carbon suitable for catalytic oxydehydrogenation of alkyl aromatic compounds.

Boria-alumina compositions have been described in the art for use as catalyst supports or as catalysts. For instance, U.S. Pat. No. 3,993,557 and U.S. Pat. No. 3,954,670 disclose a boria-alumina support prepared by a co-precipitation method comprising the hydrolysis of an aluminium alkoxide and a boron alkoxide in the presence of a suitable solvent and water; the obtained precipitate is filtered, dried, calcined and combined with minor amounts of catalytic material and further used as catalyst for hydrocarbon conversion processes, e.g. hydrocracking of petroleum feedstocks.

U.S. Pat. No. 5,880,051 discloses a series of boria-alumina catalysts with different range of aluminium-boron ratio, which were prepared from aluminium nitrate, boric acid, distilled water and ammonium hydroxide; the precipitate thus obtained was washed with water, dried and calcined at 600° C. These catalysts were employed in reforming of hydrocarbons.

U.S. Pat. No. 3,018,244 relates to a boria-alumina based catalyst prepared by impregnating alumina with a boron compound.

In G. Colorio et al.—“Partial oxidation of ethane over alumina-boria catalysts”, Applied Catalysis A: General 137 (1996), 55-68, alumina-boria catalysts were prepared by chemical vapour deposition and then their activity was compared in ethane to ethylene oxidation reactions with impregnated catalyst on porous and non-porous alumina.

A. Douy—“Aluminium borates: synthesis via a precipitation process and study of their formation by DSC analysis”, Solid State Sciences 7 (2005), 117-122, relates to aluminium borate catalysts synthesized via a precipitation method, in which aqueous solutions of aluminium nitrate and boric acid were precipitated into ammonium carbonate or ammonia solutions.

S. A. El-Hakam, E. A. El-Sharkawy—“Structural characterization and catalytic properties of aluminium borates-alumina catalysts”, Material Letters 36 (1998), 167-173 concerns aluminium borate-alumina catalysts prepared by a co-precipitation method, wherein aqueous solution of ammonium hydroxide was added to an aqueous mixture of aluminium nitrate and boric acid; the precipitate obtained was washed with deionized water, dried and calcined at 600-1100° C.

The use of boria-alumina catalysts in the oxidative dehydrogenation process of styrene to ethyl-benzene is also known from R. Fiedorow et al.—“Activity of alumina promoted by inorganic acids in the process of oxidative dehydrogenation of ethyl-benzene”, Bull. de I'Acad. Polonaise 8 (1978), vol. XXVI. This document discloses a process to make the catalyst by impregnating alumina with sulphuric, phosphoric or boric acid, and employing a ratio between X (X═B, S, P, Cl) and aluminium of 0.05.

Within the context of this application, the following definitions are used. Activity indicates the ability of the catalyst to convert a hydrocarbon reactant into products at specific reaction conditions used (temperature, pressure, contact time etc.). Selectivity typically refers to the amount of desired product or products obtained relative to the amount of reactant converted. More specifically, in an ethyl-benzene oxydehydrogenation process, activity commonly refers to the amount of conversion of a given ethyl-benzene charge rate, at specified reaction condition, and is typically measured on the basis of disappearance of ethyl-benzene and expressed in mole percent of ethyl-benzene charged. Selectivity is expressed as the mole percent of styrene obtained at the particular activity or reaction conditions relative to the amount of ethyl-benzene disappeared; yield is commonly stated as the moles of styrene produced divided by the moles of ethyl-benzene charged, expressed on a mole percent basis.

A drawback of the boria-alumina catalyst known from Polish J. Chem., 67, 419-423 (1993) document, which is used for ethyl-benzene oxydehydrogenation to styrene, is the low selectivity to styrene.

The object of the invention is therefore to provide a catalyst which shows improved selectivity in the oxidative dehydrogenation of alkyl aromatic or aliphatic hydrocarbons.

This object is achieved according to the invention with a process of oxydehydrogenating an alkyl aromatic hydrocarbon, wherein the boria-alumina catalyst has been prepared by a co-precipitation method comprising the steps of:

a) preparing a solution of an aluminium salt in an organic medium;

b) adding to this solution a boron compound;

c) adding ammonia gas to the mixture obtained in step b) to form a precipitate and/or a gel.

It is true that patent application EP0194828A2 already discloses a process of (oxy)dehydrogenating cumene to methylstyrene using a boria-alumina catalyst prepared by a co-precipitation method, but in this document the boria-alumina catalyst was prepared in aqueous medium, also by using ammonium hydroxide solution. In addition, this document teaches away by clearly stating that aluminium borate is a poor (oxy)dehydrogenation catalyst; for the reason that low conversion and selectivity are obtained by using this catalyst in the mentioned process.

The process according to the invention was found to show high selectivity in the oxidative dehydrogenation of alkyl aromatic hydrocarbon. Another advantage of the oxidative dehydrogenation process according to the invention is that this process can be performed without steam and at relatively low reactor temperatures, resulting in low energy consumption.

In the process according to the invention, any aromatic hydrocarbon that has at least one dehydrogenable alkyl group substituent can be used as starting compound. Suitable examples include mono-substituted aromatics such as ethyl-benzene, isopropyl-benzene, secondary-butyl benzene; di-substituted aromatics such as ethyl-toluene, diethyl-benzene, t-butyl ethyl-benzene; tri-substituted aromatics such as ethyl-xylenes; condensed ring aromatics such as ethyl-naphthalene, methyl ethyl-naphtalene, diethyl-naphthalene, and the like. A particularly preferred aromatic reactant in this reaction is ethyl-benzene, which is readily converted to the commercially important styrene.

In the process according to the invention, the oxidant employed may be pure oxygen, carbon dioxide, nitrogen oxide or air. Preferably, the oxidant is oxygen because it gives favourable selectivity. The molar ratio of oxidant to alkyl aromatic compound fed to the reactor may range from 0.1 to 10, preferably from 0.8 to 1.

The process according to the invention may be performed at temperatures higher than 400° C., preferably higher than 450° C., more preferably higher than 470° C. and most preferably higher than 475° C. Higher temperatures increase reaction rate, but too high temperature result in lower selectivity. Preferably, the reaction temperature is therefore lower than 600° C., preferably lower than 550° C., more preferably lower than 510° C.

In the process according to the invention, the contact time defined as W/F, wherein W is the catalyst weight in grams and F is the flow rate of the reaction mixture entering the reactor in ml (measured at normal conditions of pressure and temperature) per second, may be within the range from 0.2 to 1.2 g s/ml, preferably from 0.5 to 0.8 g s/ml.

The oxidative dehydrogenation reaction according to the invention may be carried out in the presence of steam or without steam. The ratio of steam to alkyl aromatic hydrocarbon may vary from 0 to 10.

The process according to the invention can be performed in various types of reactors, suitable types including a fixed-bed or a fluidized-bed reactor. The process operated in a fluidized bed reactor is preferred because it has the advantage of preventing hot spots, which can adversely affect selectivity.

The oxidative dehydrogenation process according to the invention is carried out in the presence of a boria-alumina catalyst that has been prepared by a co-precipitation method that comprises the steps of:

(a) preparing a solution of an aluminium salt in an organic medium;

(b) adding to this solution a boron compound;

(c) adding ammonia gas to the mixture obtained in step b) to form a precipitate and/or a gel;

in contrast to the impregnation of pre-formed solid alumina as used in prior art. In the co-precipitation method, a solution of an aluminium salt in an organic medium is mixed with a boron compound, and a B—Al precipitate and/or gel is formed, optionally after changing conditions or adding further compounds.

Any aluminium salt, which can be dissolved in an organic medium, can be employed in the co-precipitation method. Suitable examples are aluminium halides, hydroxides, carbonates or nitrates. Preferably, aluminium nitrate is used because it is readily available, high soluble in organic medium and gives catalyst which has high selectivity.

A solution is prepared by dissolving the aluminium salt in an organic medium. Within the context of this application, “organic medium” is understood to be a medium in which the water content is limited to the minimum amount needed to dissolve the boron salt. Any organic medium, as defined above, can be used in the co-precipitation method. Examples of organic media that can be employed in the present invention are solvents such as alcohols, ketones, such as acetone, esters such as ethyl-acetate.

Alcohols are preferred and alcohols having between 1 to 20 carbon atoms, such as ethanol, propanol, iso-propanol, n-butyl alcohol, sec-butyl alcohol, pentanol-1, pentanol-2, 3-methyl butanol-1, 2-methyl butanol-3, pentanol-3, hexanol, the various methyl pentanols, the various dimethyl butanols, the various heptyl alcohols or the various octyl alcohols are more preferred. Ethanol is the most preferred organic medium due because it is non-toxic, environmentally friendly and because aluminium salts are highly solubility in this solvent.

Suitable boron compounds for making the boria-alumina catalyst include various salts such as ammonium biborate tetrahydrate, boron alkoxides such as tri-isopropoxy boron or boric acid. The preferred boron salt is boric acid.

The boron compound may be added as a solid or as a solution, which is prepared by dissolving the boron salt in an organic solvent or alternatively in water or a water/organic solvent mixture; the water content of the resulting solution is limited to the minimum amount needed to dissolve the boron salt.

The aluminium salt solution and boron compound employed as solution or as a solid are mixed by stirring for a sufficient period of time, usually for a period of one to two hours, needed to complete the desired dissolution.

A basic gas such as ammonia or phosphine is added to the mixture in sufficient amount to form a precipitate and/or a gel. The preferred basic gas is ammonia. The precipitation and/or complete gelation occur preferably at a pH between 6 and 7.

After the reaction is complete, the precipitate or gel which has been formed may be washed, dried and subsequently calcined. The drying temperature may range from 70 to 120° C., preferably from 100 to 110, for 3 to 10 hours to ensure complete removal of solvent residues.

The calcination temperature is preferably at least 500° C., more preferably at least 600° C. or even at least 700° C.; but is preferably below 900° C., more preferably below 850° C., to result in a catalyst showing optimum performance.

The invention also relates to a co-precipitation method to make a boria-alumina catalyst, with steps and preferences as defined above.

Further, the invention relates to a boria-alumina catalyst as obtained by the above co-precipitation method. The boria-alumina catalyst shows improved behaviour in a process of oxydehydrogenating an alkyl aromatic hydrocarbon. The catalyst contains boron and aluminium in a ratio of from 0.01 to 1.0, preferably from 0.05 to 0.8, more preferably from 0.1 to 0.5 and most preferably from 0.2 to 0.3.

The invention will be further elucidated with reference to the following non-limiting experiments.

### EXAMPLE 1

73.5837 g aluminium nitrate (AN) was dissolved in 196 ml ethanol and stirred for 1 hour. Then a boric acid solution prepared by dissolving 2.4258 g boric acid in 25 ml double distilled water (DDW) was added and the mixture was stirred for 1 hour. The solution turned into a thick paste when passing ammonia gas through it and the pH was 6.2. 100 ml of ethanol was added to dissolve the paste and left overnight under reflux at about 90° C. The obtained gel was air-dried at 110-120° C. for 3 hours. Half amount of the catalyst sample was calcined at 600° C. for 20 hours (Example 1a). The other half amount of the catalyst sample was calcined at 800° C. for 20 hours (Example 1b).

### EXAMPLE 2

73.5837 g aluminium nitrate (AN) was dissolved in 196 ml of ethanol and stirred for 1 hour. Then a boric acid solution prepared by dissolving 3.0322 g boric acid in 25 ml DDW was added and the mixture was stirred for 1 hour. The solution turned into a thick paste when passing ammonia gas through it and the pH was higher than 8; glacial acetic acid was added to adjust the pH to about 6. 100 ml of ethanol was then added to dissolve the paste and left overnight under reflux at about 90° C. The obtained gel was air-dried at 110-120° C. for about 3 hours. The sample was calcined at 800° C. for 20 hours.

### Comparative Experiment A

25 g neutral alumina (Acros; with a particle size of 200 to 300 μm) was soaked in 25 ml boric acid solution (1.5161 g H₃BO₃ in 25 ml DDW) for 15 minutes followed by boiling for 2 hours. After that, the sample was dried overnight at 110° C. and calcinated at 800° C. for 6 hours.

### Comparative Experiment B

20 g aluminium isopropoxide was dissolved in 48.96 ml DDW and the mixture was stirred for 1 hour at 80-85° C. 1M HNO₃ was added until the pH was lowered from 8.3 to 3.7. The mixture was refluxed overnight at about 95° C. and air-dried at 90-95° C. for 2 hours. The sample was calcinated at 660° C. for 5 hours, and then for 2 hours at 800° C. After cooling, the obtained alumina was grinded and 2.8333 g of alumina of particle size between 0.5 to 1 mm was impregnated with boron.

For impregnation, 1.833 g alumina obtained above (particle size=0.5-1 mm) was soaked in 4 ml boric acid solution (0.1718 g H₃BO₃ in 4 ml DDW, heated to 50° C. to dissolve boric acid), followed by heating at 110 for 2 hours and drying overnight in an oven. The sample was calcined at 500° C. for 4 hours before use.

### Comparative Experiment C

187.565 g Al(NO₃)₃.9H₂O was dissolved in 250 ml double distilled water (DDW) to obtain 2M aluminium nitrate solution. 1.5458 g H₃BO₃ was added to the aluminium nitrate solution. The mixture was stirred for 1.5 hours, followed by addition of 14.31 ml acetic acid; the pH was less than 1. Concentrated ammonia solution was then added drop wise using burette. When a sol was observed to form, ammonia addition was stopped and the pH reached 6.3. The mixture was left overnight under reflux at about 95° C. After stopping refluxing, pH was 4.3. The content was then heated in air for around 2 hours until a gel was formed. The gel was dried overnight in vacuum oven at 110° C., followed by calcination at 800° C. for 5 hours.

The prepared catalysts were tested in the process of oxidative dehydrogenation of ethyl-benzene (EB) to styrene. Ethyl-benzene was fed to a reactor at a rate of 29.1 sccm and oxidatively dehydrogenated to the corresponding styrene when contacted with oxygen, in the presence of 0.25 g boria-alumina catalyst. The molar ratio of oxygen to EB was 0.9; other conditions included H₂O/EB ratio of 4:1 and contact time of 0.54 g s/ml. The catalyst samples were tested at a reactor temperature of 440 to 527° C. Selectivity data presented in Table 1 were determined after stabilization of the catalyst activity for at least 3 hours on stream. The results show much higher styrene selectivities obtained for the oxydehydrogenation process employing a boria-alumina catalyst prepared by co-precipitation in organic medium and using NH₃ gas (Examples 1a, 1b and 2) than for the oxydehydrogenation process employing a boria-alumina catalyst prepared by impregnation method (Comparative Experiments A-C). In addition, the catalyst prepared by co-precipitation using aluminium nitrate solution in water and ammonia solution (Comparative Experiment C) had much lower selectivity than the catalysts obtained by co-precipitation using ammonia nitrate in ethanol and ammonia gas (Examples 1a, 1b and 2).

TABLE 1

| | Average | | | Styrene |
|---|---|---|---|---|
| | bed temperature | Conversion [%] | | Selectivity |
| Catalyst | [° C.] | EB | O₂ | [%] |
| Example 1a | 443.8 | 55.5 | 83.0 | 65.1 |
| Example 1a | 471.2 | 65.6 | 100.0 | 68.6 |
| Example 1a | 476.8 | 56.3 | 96.8 | 90.3 |
| Example 1a | 475.1 | 46.9 | 90.3 | 94.6 |
| Example 1a | 507.1 | 47.3 | 100.0 | 84.4 |
| Example 1b | 472.7 | 58.6 | 98.4 | 69.5 |
| Example 1b | 476.1 | 45.1 | 80.1 | 97.8 |
| Example 1b | 510.0 | 53.4 | 100.0 | 89.9 |
| Example 1b | 484.2 | 54.4 | 98.4 | 96.2 |
| Example 2 | 471.5 | 63.4 | 98.5 | 74.6 |
| Comp. Exp. A | 527 | 100 | 36.1 | 30.2 |
| Comp. Exp. B | 497 | 76.9 | 39.5 | 38.1 |
| Comp. Exp. C | 465 | 100 | 82.7 | 36.1 |

1. A process of oxydehydrogenating an alkyl-substituted aromatic hydrocarbon starting compound into the corresponding alkenyl-substituted aromatic hydrocarbon product comprising a step of contacting the starting compound and an oxidant at dehydrogenating conditions in the presence of a boria-alumina catalyst prepared by a co-precipitation method comprising the steps of:
a) preparing a solution of an aluminium salt in an organic medium;
b) adding a boron compound to the solution to form a mixture;
c) adding ammonia gas to the mixture obtained in step b) to form a precipitate and/or a gel.

2. The process according to claim 1, wherein the organic medium is ethanol.

3. The process according to claim 1, wherein the aluminium salt is aluminium nitrate and the boron compound is boric acid.

4. The process according to claim 1, wherein the mixture in step (c) has a pH in the range of 6 to 7.

5. The process according to claim 1, further comprising a step of calcining said precipitate at a temperature of 600-850° C.

6. The process according to claim to claim 1, wherein the starting compound is ethyl-benzene and the product is styrene.

7. The process according to claim 1, wherein the oxidant is oxygen.

8. The process according to claim 1, wherein contacting the starting compound and the oxidant is at a temperature of from 475 to 510° C.

9. A co-precipitation method to make a boria-alumina catalyst comprising steps of:
a) preparing a solution of an aluminium salt in an organic medium;
b) adding a boron compound to the solution to form a mixture;
c) adding ammonia gas to the mixture obtained in step b) to form a precipitate and/or a gel.

10. The method according to claim 9, wherein the organic medium is ethanol.

11. The method according to claim 9, wherein the aluminium salt is aluminium nitrate and the boron compound is boric acid.

12. The method according to claim 9, wherein the mixture in step (c) has a pH in the range of 6 to 7.

13. The method according to claim 9, further comprising a step of calcining said precipitate at a temperature of 600-850° C.

14. A boria-alumina catalyst prepared by a co-precipitation method comprising:
a) preparing a solution of an aluminium salt in an organic medium;
b) adding a boron compound to the solution to form a mixture;
c) adding ammonia gas to the mixture obtained in step b) to form a precipitate and/or a gel.

15. The boria-alumina catalyst according to claim 14, containing boron and aluminium in a ratio of from 0.1 to 1.0.
